(19) United States
(12) Patent Application Publication
(10) Pub. No.: US 2007/0168225 A1
(43) Pub. Date: Jul. 19, 2007

(54) Workflow generator for medical-clinical facilities

(76) Inventors: Sultan Haider, Erlangen (DE); Michael Mankopf, Mohrendorf (DE); Henning Schneider, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 11/603,767

(22) Filed: Nov. 22, 2006

(30) Foreign Application Priority Data
Nov. 24, 2005 (DE) 10 2005 056 081.4

(51) Int. Cl. G06Q 10/00; A61B 5/00

(52) U.S. Cl. 705/2; 600/300; 128/920

(57) ABSTRACT

In a method, a system and a central control device for generation of a workflow for a medical treatment of a patient, the treatment is divided into treatment segments that are implemented on a number of instances. After the acquisition of a symptom list, a set of diagnostic questions is generated that are answered by responses. An optimized, case-specific workflow based on the symptom list is thereupon automatically generated.

### BACKGROUND OF THE INVENTION

1. Field of the Invention

The present invention is in the field of medical technology and in particular concerns workflow planning of medical, diagnostic or other therapeutic actions that typically include a number of treatment steps, the treatment steps being implemented at a number of possibly different technical apparatuses and possibly also in different clinical facilities.

2. Description of the Prior Art

There has previously been no complete solution for a total workflow planning of a treatment of a patient.

The current procedures in conventional systems can be described as follows in the example of a medical emergency.

In a medical emergency, the patient or a relative notifies an emergency physician or the ambulance. The emergency physician analyzes the state of the patient and (in the event that it is possible) generates an initial emergency diagnosis. The physician typically asks (via telephone) different clinical facilities and hospitals whether the necessary clinical resources (such as, for example, operating room or heart catheter laboratory, further technical apparatuses for medical emergency care) are available. If necessary, the emergency doctor also administers suitable drugs. After the arrival at the selected clinical facility, the doctors on duty are informed by the emergency physician about the incoming case, about the preliminary diagnosis, the pre-history, already-initiated therapeutic actions or administered medicines or other measures. The accepting physician then normally decides as to further treatment and/or examination steps as well as with a regard to the need for medicine supply, such as a drip feed.

In conventional systems the interface between the emergency physician and the selected clinical facility is a manual or verbal interface. In other words, the data already acquired by the emergency physician are normally relayed via written emergency physician protocols to a further person in the clinical facility who then acquires the respective data himself or herself, or indirectly via yet a further person and (possibly) inputs this data into an electronic system. This possibly repeated and partially manual transfer of the data is error-prone.

After the arrival at the medical facility, complex medical procedures are normally executed for the purposes of a medical diagnosis. Since little to no information whatsoever is known and present with regard to the current emergency, multiple further examination procedures are typically initiated in order to be able to collect as much information as possible about the emergency and with regard to the patient. These initiated medical actions include laboratory tests, imaging methods (such as examinations with an ultrasound apparatus, a magnetic resonance apparatus, a computed tomography apparatus etc.), ECG and/or EEG examinations and the like. The responsible physician forms a picture of the state of the patient based on the detected symptoms and newly, individually establishes (respectively for each patient) which further steps should be introduced. Depending on which steps he or she has established, the medical resources required for this must be checked for availability. This has previously also not ensued in an automated manner. In addition to the medical personnel, the medical resources are, for example, technical devices such as the apparatuses cited above, available medicines, etc. It is frequently necessary that examinations must be conducted at different modalities (for example at an ultrasound apparatus and at a magnetic resonance apparatus). It is frequently the case that this process ensues in two stages, with an examination ensuing in a first step, at a first modality, and in a subsequent second step or in a second state, a further examination ensues at a second modality in the event that the results of the first modality were not satisfactory or were not significant enough. In these cases further availability inquiries, in particular with regard to the necessary technical devices, must be initiated.

The procedure described above according to the prior art is disadvantageously error-prone, very time-consuming and frequently leads to a non-optimally designed treatment workflow since treatments and/or examinations may be executed repeatedly in part, although not necessary or because other examinations that would be necessary are possibly unavailable.

### SUMMARY OF THE INVENTION

An object of the present invention is to optimize and automate workflow planning in the framework of a medical treatment of a patient and thereby increase the efficiency of the entire treatment process. Moreover, a centralized control of the entire clinical workflow should be possible.

As used herein, a “workflow” is a workflow plan in the framework of a medical-therapeutic treatment of a patient. The workflow is primarily based on digital data and can be processed via a computer-based system. It is likewise possible to take into account further data sets that have been acquired via manual input via a user interface or that have been acquired automatically via a system interface with medical apparatuses such as, for example, laboratory systems, ECG etc. A workflow typically involves a complex medical progression that can be organized (compartmentalized or divided) in different phases beginning, for example, with an emergency and ending, for example, with the release from the clinical facility. In alternative embodiments it is likewise possible for the workflow to include only one specific region from this complete progression with regard to the patient. Furthermore, it is normally the case that the workflow involves different clinical and/or therapeutic or other facilities that are necessary in the treatment of the patient. It is likewise possible to limit the workflow to only one configurable selection of clinical facilities. For example, it is possible for the workflow to be generated to be limited to only one specific clinical facility. However, typically all medical actions that are necessary in the framework of the treatment of the patient are encompassed by the workflow. Since the workflow serves to control the entire treatment progression of the patient, it must be available at every point in time of the treatment. It is therefore equipped with corresponding interfaces so that it can be recalled at any time by authorized personnel.

As used herein, “medical treatment” encompasses all medical, therapeutic (possibly medicinal-therapeutic) and/or all other procedures that can be executed in the framework of administering medical care. They include the treatment by a doctor, technical treatments via medical apparatuses, examinations by a different doctor (including laboratory examinations) as well as clinical processes or other therapeutic applications. Furthermore, such methods and processes that are not directly relevant but rather can be indirectly obliquely relevant in the framework of a medical treatment, and encompassed under the term “treatment”, such as operational methods, organizational processes or other processes that can be processed in a computer-based digital system and be of relevance in health care. Furthermore, in this context it is noted that a treatment in the sense of this invention does not necessarily have to be executed in a clinical facility or, respectively, in a hospital; rather, it can also be executed by an established (resident) physician, a specialist physician, an emergency physician or by other persons active in health care.

In principle there are different possibilities as to how the treatment is executed. It is possible for a doctor to execute the treatment so to speak manually (thus directly on the patient), for example an orthopedic treatment or a manual examination. Moreover, it is possible for the treatment to ensue in the framework of an exercise, electrophotographic a physiotherapeutic exercise. A number of treatments can moreover be executed at medical-technical apparatuses.

An “instance” is characterized by the potential for implementation of the treatment of the patient. In the sense of this invention an instance can concern both a facility and an apparatus. The term “instance” in the framework of this invention is not limited to a specific facility, but rather refers to all medical-technical apparatuses or to more complex facilities up to a clinical facility. An instance also can involve all modalities such as, for example, ultrasound apparatuses, x-ray apparatuses, computed tomography apparatuses, magnetic resonance apparatuses as well as laboratory technical systems, etc.

The present invention is subsequently described using the solution according to the method. The advantages thereby mentioned, alternative embodiments as well as advantageous features are correspondingly also applicable to the other solutions of the object, in particular to the inventive centralized control device.

The invention is not limited to the specified order (sequence) of the method steps. It likewise lies within the scope of this invention to execute the steps in a different order, partially in parallel or temporally overlapping (in an interleaved mode).

The inventive method serves for automatic generation and/or control of a workflow in the framework of a medical treatment and/or examination. Typically the inventively generated workflow is executed automatically.

According to the invention, a medical workflow system is provided that serves to direct employees (staffers) in health care via a central computer-assisted method. The employees can be medical personnel (in particular physicians or nurses) and administration employees or other personnel in the framework of an outsourcing.

The workflow is inventively generated automatically and case-specifically in order to optimize the entire process in the framework of the medical, diagnostic and/or therapeutic treatment and/or to be able to also take into account economic or financial aspects in an optimized manner. The inventive method uses an input/output mechanism, a databank (in particular a rule database) that make the system a self-learning system with probability estimations and probability-based prediction, training and optimization algorithms (such as, for example, the “back propagation algorithm” as a learning (training) algorithm or other mechanisms, as well as with adaptive filter techniques, Bayesian techniques, genetic algorithms, etc. These mechanisms and techniques are used in connection with the central databank in which data sets with regard to the patient are stored in digital form and in which what is known as a workflow generator is programmed and/or executed.

With the aid of the inventive method the clinical personnel are directed in the implementation of the treatment by means of the automatically-generated wbrkflow, by sequential steps being proposed so that a sequence of treatment segments is generated that should ensue in the framework of the medical treatment of the patient. This process includes an automatic planning method known as a “scheduling” for date planning and booking, an automatic structuring of the relevant data, a documentation and a communication with the users via a user interface. According to the invention, the automatically-generated workflow includes a sequence of multiple diagnosis and treatment segments, typically with later treatment segments build on one or more treatment segments executed earlier, or being based on these earlier segments.

According to the invention the workflow is automatically generated based on specific parameters. The parameters are advantageously initial symptom lists and responses to diagnostic questions. In this embodiment two categories of parameters are provided, namely symptoms and diagnostic answers. In alternative developments, it is possible to additionally take into account further parameters or to limit the parameters to the parameters mentioned above. Moreover it is possible to replace the parameters mentioned above with other parameters.

For example, if the response to a specific diagnostic question is based on a laboratory test to be executed, it is necessary for the laboratory test to be executed chronologically before the response to the diagnostic question in order to be able to provide the necessary information. An optimized temporal planning or scheduling of the treatment progression as the invention proposes is therefore necessary. In a preferred embodiment, it is provided that the proposals for the sequence of treatment segments are based on predefined workflows. It is also possible for the proposals for the sequence of treatment segments not to be based on predefined workflows, but rather the proposals are generated for the first time and in a wholly novel manner based on other parameters. Incorporated into the generation of the workflow are, among other things: the clinical state of the patient (which is typically input via specific checklists), the current situation (in particular the availability of medical modalities, personnel or other equipment) and the experience (as a calculation of probabilities, since the system is or can be fashioned as a self-learning system and thus learns from the previously-generated workflows, such that this knowledge can be taken into account again given later workflows to be generated).

In the preferred embodiment the workflow to be generated is constructed upon delivery of a patient to a clinical facility. An initial symptom list and a set of responses to a set of diagnostic questions are therefore taken into account as parameters. Upon the delivery of the patient determined specific symptoms are typically recorded. These symptoms are stored in the system in what is known as an “initial symptom list” of the patient. In a simple design of the inventive solution, the initial symptom list remains constant during the execution of the inventive method. It is thus no longer altered. In a more complex exemplary embodiment of the invention, the initial symptom list can be expanded (supplemented) at a later point in time (if applicable, also at any time) with further symptoms, in particular with such symptoms that arise or become noted in the framework of the medical treatment of the patient.

Parallel to the acquisition of the symptom list or at a later point in time, a set of diagnostic questions is typically automatically generated with regard to the respective patient. The diagnostic questions are advantageously defined and generated with regard to the detected symptoms. For example, if a patient is admitted with a suspicion of an acute heart infarct, in this state in accordance with the invention irrelevant diagnostic questions (for example, orthopedic questions) are removed. It is normally the case that the definition of the diagnostic questions ensues relative to the symptoms, however, still further parameters can alternatively be taken into account here.

After the definition or generation of the diagnostic questions, a process is introduced for responding to these diagnostic questions. This response process can be designed in different manners. It is possible for the response to ensue by the treating doctor via a user interface. It is also possible for the response to make further examinations or diagnostic methods necessary the latter case, according to the invention the respective examinations are triggered automatically or semi-automatically. Diagnostic questions are not only verbal questions to the patient but also all data that are surveyed, for example laboratory data, x-ray images, ultrasound images, results from chemical or biotechnological tests etc. These data can also be automatically imported via system interfaces.

After all results for response to all diagnostic questions are present, the response process can be concluded. The system thus records all answers to the diagnostic questions. After the conclusion of the response process, dual use is made of the data so collected. The are stored in a databank of the system such that this knowledge is available for the later workflow generation. Additionally, this knowledge is used in order to automatically generate the case-specific workflow. The generation of the workflow ensues in that the initial symptom list and/or the answers to the diagnostic questions are processed.

In an embodiment of the invention, the generation of the workflow includes still further parameters in addition to the initial symptom list and in addition to the responses to the diagnostic questions.

The preceding described processes thus refers to the generation of the workflow. They can also be executed via a workflow generator.

It is typically the case that the generated workflow is subsequently also executed automatically and/or semi-automatically. In order to increase the security of the system, in alternative embodiments it can be provided that the generated workflow is not executed automatically but rather that its execution depends on further authorization checks. In this case, an authorized person receives the generated workflow as a suggestion and must effectively “using off” on this. In this case the medical competency of a selected person is ranked higher than the competency of the inventive automated system. However, this feature is optional.

Since the generated workflow concerns the entire medical treatment process, it can also be provided to execute only parts of the generated workflow. This feature concerns, for example, cases in which the workflow represents a total treatment plan inclusive of emergency measures, further treatment in the clinical facility as well as rehabilitation measures. At the clinical facility, only the part of the workflow is then executed that concerns the respective clinical facility. Later rehabilitation measures that are executed, for example, by a different facility are not automatically activated.

In order to design the system to be as transparent as possible, the generated workflow is visually displayed. The clinical personnel are thus informed about the generated workflow. Nevertheless, if the workflow should turn out to be deficient for specific reasons, in an embodiment the automatically-generated workflow can be manually modified via specific control mechanisms. This typically ensues via a specific user interface.

Since the knowledge standard continuously rises in the medical sector, the inventive method can advantageously take into account new knowledge, since the method is dynamically designed. For example, if previously a specific symptom list and/or responses to specific diagnostic questions led to the treatment of the respective patient according to scheme A and this procedure has changed due to the new knowledge, according to the invention it is thus possible that a treatment concept according to scheme B is now initiated given the same conditions. The inventive method is thus not static but can take into account novel treatment concepts.

By contrast, it can be the case that a novel treatment concept turns out to be deficient and a previous treatment concept must be returned to. In this case it is necessary to be able to access an older treatment concept and therewith an older workflow. It is therefore provided to store the respective generated workflows or at least portions thereof, in a log file or in a databank in order for them, to be available at a later point in time. Errors in the generation of the workflow can thereby also be dealt with and remedied more easily.

A further advantage of the inventive solution is that the treatment is centrally planned and/or is controlled end that the scope of the inventive method can be adjusted dynamically. It can thus be set that the workflow to be generated is in principle wholly comprehensive and involves the entire treatment process of a patient, including the current treatment as well as further follow-up treatments or corresponding rehabilitation measures. This has the advantage that a treatment concept for a patient can be executed on a very abstract level and at a central location. Given a decision process in the framework of the medical treatment of the patient, factors thus can also be additionally taken into account that would have possibly have been disregarded given a purely manual procedure, as was previously the case in the prior art. However, in a simpler embodiment of the invention the workflow can be limited to a specific clinical facility or to a treatment segment.

According to the invention a centralized control instance is provided that is designed to plan a complex treatment process of a patient in the framework of a medical examination and, if applicable, to automatically and/or semi-automatically execute the treatment process. The advantage of the control on a central plane is (as mentioned in the preceding) to be seen in that optimally all factors that are relevant in the framework of the medical treatment of the patient can be taken into account as well. For example it is possible that, given an emergency acquisition, data sets can also be accessed that concern an earlier treatment of the same patient or the parallel treatments of the patient. So that this can be enabled, all instances that are necessary in the framework of the medical treatment of the patient are involved in data exchange with one another. This can be a data exchange via wireless interfaces.

A further advantage of the inventive method is that all results or partial results of the method are stored in a central databank and thus are also available to other processes or at a later point in time. Moreover it is also possible to track modifications of treatment workflows over a time span. It is, thus possible to detect how a specific treatment workflow has been planned earlier and how it is planned now in comparison.

The method is normally designed such that it ensues automatically or semi-automatically. Errors due to incorrect manual inputs can therewith be reduced. Given the semi-automatic execution, it is provided that suggestions that the user can then select are offered to him via the user interface. However, the generation of the diagnostic questions and the generation of the workflow ensue automatically according to the invention.

The result of the inventive method is the generation of a mask or a template that in particular relates to the category or the type of the application case. The workflow includes a series of treatment segments that are matched to one another and designed to be optimal for the respective case. Moreover, a clinical report to the physician can already be designed with regard to the particular case, with an optimized display that relates to the respective examination results, symptoms and/or responses to the diagnostic questions. Moreover, further functionalities can be offered that are necessary in order to further process the symptoms, the responses to the diagnostic questions etc. or to present these in a suitable manner. For example, if it was necessary to generate an x-ray image given the response to a diagnostic question, according to the invention it is thus automatically provided that the functionality is made available for display of the x-ray image.

Moreover, an authorization module can be provided that is designated to execute a check of the authorization of the user. With this feature the safety of the system can be increased in that the free availability of results is limited to a specific circle of persons in order to avoid a misuse.

In a preferred embodiment, emergency care devices are also connected to the inventive system. In this case, two variants are provided according to the invention:
Variant 1: the patient notifies the emergency physician, who is then connected to the inventive system and is supported by the inventive method.
Variant 2: the patient notifies the emergency physician only indirectly via a call center. The call center is then connected to the inventive system and is directed (controlled) by the inventive system.

In the first variant, all emergency physicians thus possess a mobile, wearable device that is involved in data exchange with the central workflow instance. In the case of emergency, an emergency an emergency physician can thus also be supported by the inventive system. For example, after he or she has input the current symptoms of the emergency patient and, if applicable, has answered further diagnostic questions, a process can be started that selects the nearest facility for treatment of the respective patient. In this embodiment, it is normally not necessary for the mobile, wearable devices of the emergency physicians to be equipped with a location determination device since the control center is informed about their position anyway. However, in alternative embodiments the devices can also be equipped with a GPS receiver or the like. After the determination of the current location of the treating physician (and thus the location of the emergency patient), the nearest facility can be found. In the search for the nearest facility further parameters can be checked that are necessary for treatment of the patient. For example, if the emergency patient must undergo an immediate emergency operation, in the search for the nearest medical facility it can simultaneously be checked whether the required medical instances are available. A query is thus automatically started at the clinical facilities as to whether the operating room is free in the relevant time span, whether diagnostic modalities are available and/or whether the required medical personnel are available. According to the invention, all of these queries are generated automatically. The emergency physician thus is spared time-consuming and error-prone manual queries that previously normally ensued via telephone. The entire method can be distinctly improved and accelerated.

Corresponding considerations apply with regard to the second variant, wherein the call center is controlled with the inventive method and is designed to ensure an optimally selected instance for primary care for the patient. The instance can be an emergency physician (the method can then proceed according to variant 1) or a clinical facility can be directly determined that is optimally equipped for the patient at the current point in time.

After a clinical facility that is optimally equipped for the current needs has been found, the workflow includes further treatment steps that must be executed in this medical facility in the framework of the medical treatment. Here as well the overall medical process can clearly be improved in accordance with the invention.

According to the invention, post-treatment segments are also encompassed in the workflow, such post-treatment segments are executed after the current treatment (such as, for example, rehabilitation measures or follow-up operations or follow-up treatments in the same or another clinical facility. In these cases, the relevant data sets that have previously been collected with regard to the patients are automatically relayed to the relevant facility. This achieves the further advantage that the relevant information is provided securely and reliably. In the manual procedure according to the prior art it is frequently, disadvantageously the case that prior examination results or other information are lost and are no longer available at a later point in time. This leads to the situation of the patient being unnecessarily subjected to a repeated examination. This disadvantage can be inventively avoided.

The inventive embodiments of the method described in the preceding can also be embodied in a computer program product (computer readable medium encoded with a data structure), that causes the computer or processor in which it is loaded to implement the inventive method described above by the data structure being executed by the processor as program code.

Individual components of the method described in the preceding can be executed as described units and the other components can be executed in another unit, the units being individually sold as a distributed system.

### DESCRIPTION OF THE DRAWINGS

FIG. 1 is a flowchart of an example of a workflow according to the inventive method, in a preferred embodiment.

FIG. 2 schematically shows parameters for the inventive method according to the preferred embodiment.

FIG. 3 is an overview representation of instances that can be used in the inventive method.

### DESCRIPTION OF THE PREFERRED EMBODIMENTS

The present invention describes a complete solution for planning of a medical treatment of a patient. For planning, the treatment a workflow is generated that is designed optimized to the respective case of the patient.

In the preferred embodiment the generated workflow is based on an initial symptom list S of the patient that was defined by an emergency physician or by an admitting physician.

In a preferred embodiment of the invention this is applied for emergency treatments. In this case the previously manual method can be distinctly accelerated and improved by an automatic planning of the necessary medical treatment that ensues automatically a sequence of different treatment segments. In particular a query for availability of necessary instances I for execution of individual treatment segments is automatically triggered. It is thus no longer necessary for the doctor to manually request specific resources such as intensive care beds, corresponding resources in the clinical facility etc. Moreover, multiple (and thus unnecessarily repeated) queries in connection with the determination of the state of the patient can be avoided in that an initial symptom list S is detected once and in that the workflow is generated based on the acquired symptom list S. In a first embodiment of the invention, the emergency physician is connected to the inventive system via a suitable interface so that he or she already inputs the symptoms in the form of a symptom list S via a mobile handheld device upon an acute emergency treatment of the patient. The recorded symptoms are thus already present in the system and do not have to be input again at a later point in time, as in previous systems. The symptom list S is also already available given an admittance of the patient into a clinical facility.

In a second embodiment of the invention, the medical emergency care or another care of a patient is switched in advance to a call center that is controlled by the inventive method.

The present invention is comprehensively designed and concerns not only the planning of a medical treatment for emergency patients but also any medical, diagnostic and/or therapeutic treatments of a patient. The scope of the inventive method is dynamically configurable. It can thus be set which instances I should be taken into account in the method. For example, it can be set whether the method should be limited only to such instances I that are relevant in the framework of an emergency treatment (such as, for example, emergency physician, clinical outpatient facilities, operating room) or whether the method should also be comprehensively used in a larger scope so that follow-up treatments should be included such as, for example, rehabilitation measures, further diagnostic examinations, etc.

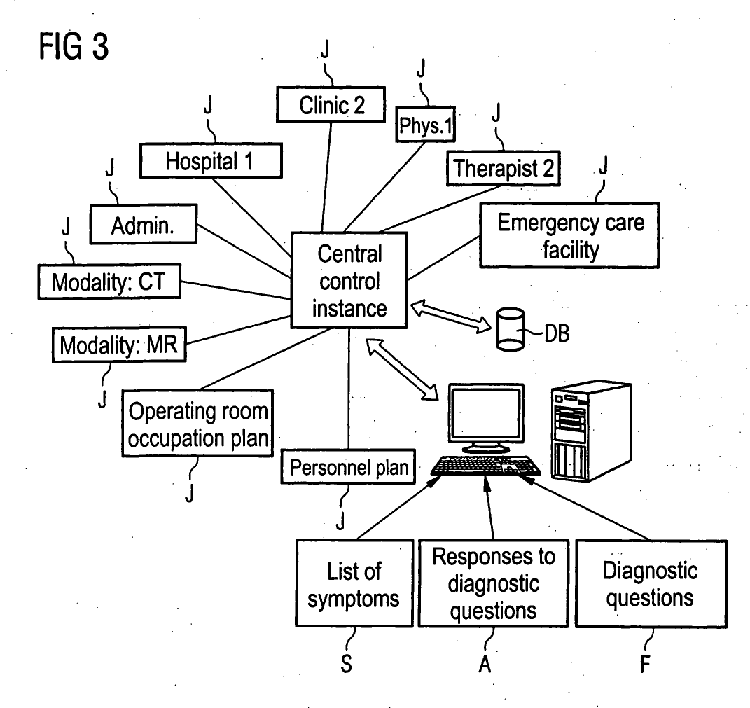

FIG. 3 schematically shows that—depending on the configured scope of the method—all instances I to be considered are involved in data exchange with a central control instance. The instances I shown in FIG. 3 are listed only as examples. They can be replaced at any time by further instances or other instances can be added. It should be noted that the instances I do not have to be exclusively medical instances; but also can include such instances as are relevant in the framework of an economic or financial evaluation of a medical treatment of the patient. Moreover, further factors that should be considered in the framework of the medical treatment of the patient have an influence on the generation of the workflow. A particular advantage of the invention is that the scope of the inventive method can be dynamically configured.

Moreover, the inventive system is very flexible since the instances I can include many different facilities and do not necessary have to lie on the same hierarchical plane of the system. For example, it is possible that an instance I concerns a clinical facility, a specific modality (such as, for example, a CT apparatus or an MR apparatus, etc.), an assignment and/or personnel plan for the operating room, a specific established physician, a pool of established therapists and/or physicians etc. In the framework of the invention, a set (quantity) of instances that are available in principle is provided, which instances I can be embodied in the inventive method depending on the adjustment of the scope thereof. All instances I from the set mentioned above are thus involved in data exchange with a central control instance. With regard to communication technology, they are further involved in data exchange with one another and with a central databank DB via the central control instance. All data of the respective instances I and the patients treated and to be treated are stored in the databank DB. Moreover, all results of the inventive method or, respectively, all intermediate results and in particular the generated workflow are likewise stored in the databank DB.

Via the inventive central control on a hierarchically superordinate plane, it is possible for a complex medical treatment that includes different treatment segments to be planned in an optimized manner. The optimization criteria can be advantageously, dynamically configured. For example, a user can set whether the generated workflow or the treatment planning should ensue optimally quickly. In this case the optimization criteria concerns the time factor. Other arbitrary optimization criteria can also be set cumulatively or alternatively, for example an optimization with regard to the incurred costs for the patient, an optimization with regard to the costs for the clinical facility, an optimization with regard to the spatial conditions so that the patient should optimally be treated in a spatially limited environment, etc.

The central control instance shown in FIG. 3 is assigned to generate the workflow. This ensues with access to the databank DB and, if applicable, with access to a user interface. Via the user interface a user (in particular a treating doctor) can input further parameters that should be used for generation of the workflow. Moreover, he can be supported via a specific design of the user interface in that a selection of possible decision alternatives is offered to him or her given a decision to be made. It is typically necessary for a user to be authorized in the system in order to avoid a misuse.

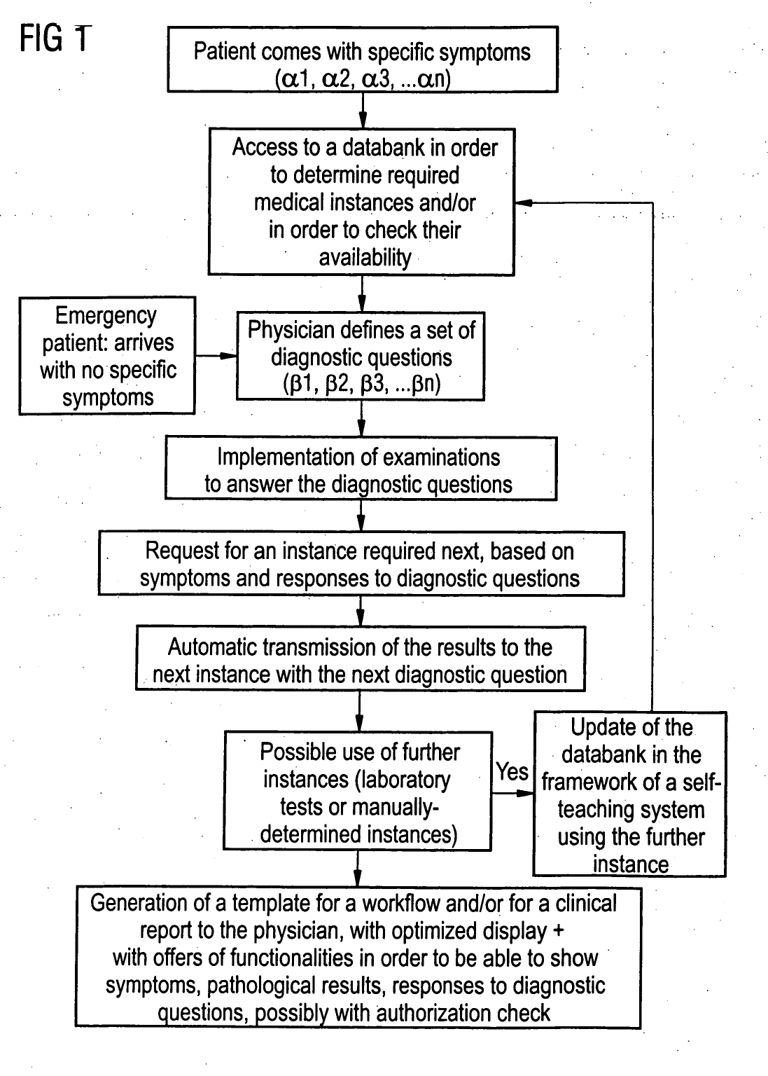

In the following a workflow of the inventive method according to a preferred embodiment is described in connection with FIG. 1.

An emergency physician is notified due to an emergency of the patient. The patient or the patient's relatives or caregivers inform the emergency physician about the symptoms of the patient.

In an alternative embodiment the preceding described procedure is modified somewhat in that an emergency physician is not directly notified, but instead a call center agent is first indirectly notified of a treatment of the patient. The call center can be designed for an emergency treatment of a patient or for a normal treatment of the patient. The call center is connected with the central control instance and is controlled thereby. It includes a call center agent for data exchange with the user, here with the emergency patient or his or her relatives. The call center agent thus poses questions and receives the responses. The call center agent is directed by the inventive central control instance through a structured question catalog. The patient specifies a list of symptoms S. Based on the symptom list S acquired by the call center, the call center automatically generates a set of diagnostic questions F to be answered by the patient. Dependent on the responses of the patient, further diagnostic questions F are possibly output that must in turn be answered on the part of the patient. The patient thus specifies a response A to a diagnostic question F, which response is recorded by the system and further processed. Overall an optimal medical care service with the required resources can be provided based on the sequence of diagnostic questions F and responses A to the diagnostic questions F, which medical care service is then responsible for the respective emergency.

According to the response to the diagnostic questions F, the system selects automatically-determined emergency facilities or hospitals. The hospitals selected here are typically structured according to specific criteria and output in the form of a list. A ranking of the selected hospitals thereby ensues according to configurable criteria. The location or, respectively, the proximity to the emergency is typically used as an optimization criterion, however, other criteria are also alternatively or cumulatively conceivable. The nearest hospital thus occupies the first position in the list, followed by further hospitals at a further spatial distance. A query to the facility standing at the first position of the list is thereupon initiated, which query checks an availability of the necessary resources in this hospital. For example, at this point it is checked whether an operating room is available in the hospital in a relevant time span, whether the medical personnel are on duty and—in the event that further examinations are necessary—whether the respective modalities are available. In the event that all checks were successful, this hospital is selected. Otherwise (when, for example, the necessary resources are not available in this hospital) the method described in the preceding is executed with regard to the next hospital in the list. This procedure is repeated until a clinical facility can be found for treatment of the patient, which clinical facility is then offered by the agent of the call center.

In the procedure described in the preceding for selection of a suitable medical facility for treatment of a patient, the inventive method is applied at a call center, the call center being designed for selection of a suitable facility for medical care of a patient. It is not necessary for the call center to be designed for emergency treatment of patients. In principle it can likewise be activated before utilization of a medical service. The call center is controlled by the central control instance and/or according to the features of the inventive method.

If no call center should be used, the inventive method can be used according to the previous scheme.

These steps are shown in general in the first three boxes in FIG. 1. The specific symptoms α1, α2, α3, . . . , αn of a patient are recorded in a symptom list S. The recording ensues either via the treating physician or via the agents of the call center. After the acquisition of the symptom list S an access ensues to the databank DB. With regard to the acquired symptom list S a set of diagnostic questions F (designated in FIG. 1 with, for example, β1, β2, β3, . . . ,) is generated. At this point two variants are again provided.
the diagnostic questions F are directly defined or modified by the physician himself or herself or are selected by means of a semi-automatic selection, or
a set of questions F is automatically generated by the system, in particular by the central control instance under access to the databank DB.

In some cases, to answer a diagnostic question F it is necessary that still further medical examinations must be conducted. In this case it is provided that the further medical examinations are either executed directly or at least initiated. This can ensue, for example, by a specific notification to the corresponding modality (for example given an MR apparatus).

All necessary examinations are conducted to answer the diagnostic questions F. This typically ensues sequentially. In this case the data collected beforehand are forwarded by an examination instance I to the respective next examination instance I until all examinations can be concluded. However, it is also possible that individual examinations are executed in parallel. In the event that this is possible, this is initiated in order to save time. A parallel execution can be indicated, for example, based on laboratory tests.

After conclusion of all necessary examinations it is possible to answer all diagnostic questions F.

Given the response to the diagnostic questions F two alternatives are proposed in turn:
the questions F can be manually answered by the physician in that he inputs the responses A via a specific user interface, or
the response to the questions ensues semi-automatically in that a selection of possible answers A to the diagnostic questions F is offered to the physician, from which selection he then must select via a suitable menu on the user interface.

After all responses A to the diagnostic questions F have been acquired by the system, it is inventively provided that a workflow respectively optimized for the respective case can be generated. This ensues either automatically or semi-automatically via a corresponding selection of suggestions on the part of the user.

In the preferred embodiment of the invention, the generation of the workflow is based on the initial symptom list S and on the responses A with regard to the diagnostic questions F. This has the advantage that a decision about the workflow can be made relatively quickly and simply. In simpler embodiments of the invention, the generation of the workflow is based only on the symptom list (for example, when the state is unambiguous and no further questions are to be clarified) or only on the answers A (for example since the answers A are indirectly related to the symptom list S).

In alternative embodiments still further parameters in addition to the symptom list S and the answers A are cumulatively or alternatively considered. For example, it is thus possible that the initial symptom list S is modified or, respectively, expanded by further symptoms collected in the course of time. In this case a modified symptom list is used. Moreover, it is possible, for example, to consider still further manual inputs of the physician that concern special factors (such as, for example, allergies, etc.).

In an embodiment of the invention, an optimized structuring of the data is offered in the generation of the workflow. This means that specific templates or sample patterns are generated for the clinical report of the physician. The generated templates are advantageously based on a computer-based electronic processing and are offered via a corresponding user interface. The generated template thereby includes an optimized structuring of the data with the display areas for texts, images, videos, voice messages that are relevant for the respective case. Moreover, the corresponding functionalities for representation of results are provided. If the generated workflow comprises, for example, an x-ray examination but no laboratory test, the data are automatically structured in the generated template such that the input of an x-ray finding is provided while laboratory results are not taken into account. The generated template is specifically designed (and therefore optimized) for the workflow. The functionalities for display of the results or intermediate results are also correspondingly generated in the framework of the workflow. In the example cited in the preceding, the functionality is thus provided for display of x-ray examinations. The templates that must be filled out in the framework of the treatment of the patient in accordance with the invention can thus be generated very brief, simple and case-specific.

In principle it is also possible for the inventive method for generation of a workflow in the framework of a medical treatment of a patient to be used multiple times. For example, the method can be used in a first step A in order to find an emergency facility for care of the patient. In the event that such a clinical facility has been found, the method can be reapplied in a step B in order to generate an optimally adapted workflow for treatment of the patient in this clinic. In this case, after arrival in the clinical facility the physician analyzes the symptom list S of the patient and initially drafts an initial diagnostic evaluation of the case for the purpose of the emergency treatment. This initial diagnostic evaluation may be, for example, the diagnosis of an acute myocardial infarct. The physician makes a corresponding input into the system via the user interface. This can ensue either manually or supported in that the physician selects a selection of offered options on the user interface. The central control instance thereupon automatically prepares a set of diagnostic questions F that must be answered for treatment of the patient. This advantageously ensues with access to the databank DB.

In an advantageous development of the invention, it is provided that still further suggestions are additionally offered in addition to the diagnostic questions F, which further suggestions concern, for example, the current treatment of the patient. At this point, for example, steps can be suggested that concern a medication of the patient, an examination to be conducted in parallel such as, for example, an EEG etc. In the case of a preliminary diagnosis “heart infarct” (as described in the preceding example), the results so collected can be automatically, electronically relayed to the respective department of the hospital in order, for example, to be able to implement a heart catheter examination or, respectively, treatment.

The central control instance can be used for different purposes with the method proposed here. The input of data is thereby advantageously very limited. It is merely necessary to input a list of initial symptoms S and—in the event that this does not ensue automatically—to answer specific diagnostic questions F.

The inventive central control instance includes the following functionalities:
it seeks the next facility (clinic, hospital, privately established physician, other facility for execution of diagnostic examinations etc.) for treatment of the patient;
it checks the availability of necessary instances that are necessary for treatment of the patient;
it in particular checks the availability of operating room, intensive station and, for example, in particular facilities for implementation of a heart catheter examination;
it checks the availability of modalities such as MR, CT, AX;
it automatically suggests an optimized workflow for treatment and/or for further diagnosis of the patient after the suitable facility has been found. At this point different optimization criteria can be used in the framework of the optimization of the workflow. As already explained, the primary optimization criterion is the time. For example, it is therefore typically proposed that it is better to use a troponin fast test instead of a significantly more elaborate laboratory test with a complete scale;
it offers an optimization of the workflow or of the treatment plan under consideration of the acquired symptom list S and/or the answers A to the diagnostic questions F that have possibly been answered by examinations;
it offers a consideration of new medical knowledge in that modifications of previous workflows are detected directly and immediately and have as a result an update in the databank DB. In this context it is provided that an authorized physician can modify the previous method via a corresponding manual input and can in particular modify automatic workflows. The modifications that have occurred in such a manner are automatically accounted for in the future workflows. New medical knowledge can, for example, lead to the situation that the previous set of diagnostic questions F is no longer generated with regard to a specific symptom, but rather an additional question must be posed that leads to a modified set of diagnostic questions F′. The modified diagnostic question set F′ is then used with regard to the respective symptom in all future method workflows;

new treatment concepts, methods and other improvements can be immediately considered via an update in the databank DB;

all information relevant for the treatment of the patient are available at a central location and can, if applicable, be automatically related to further facilities without having to fear an information loss. Moreover, the planning or the introduction of further steps is possible.

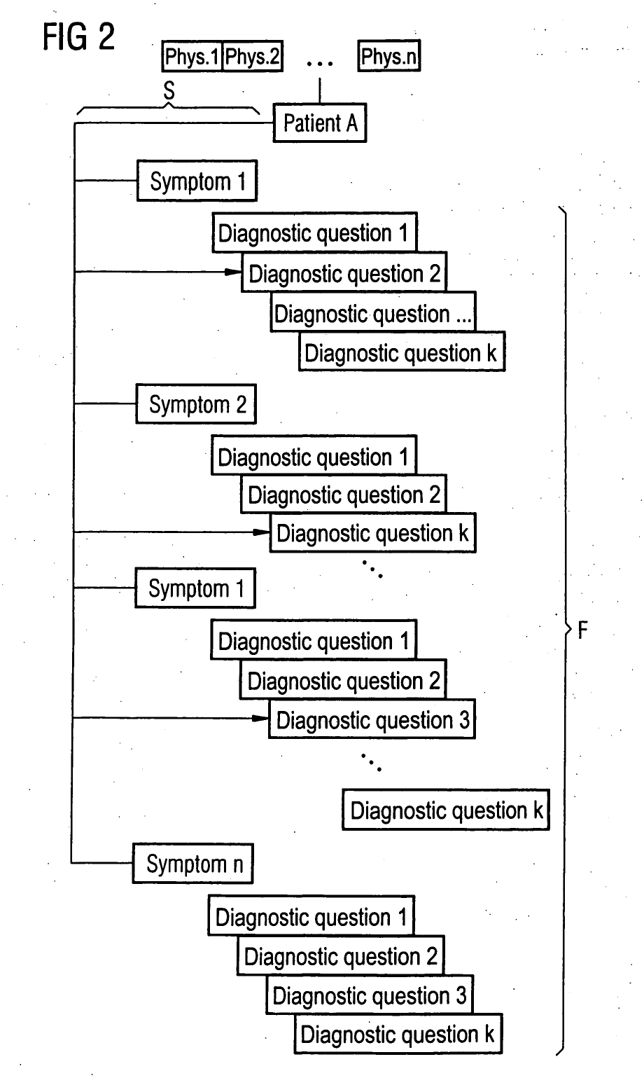

FIG. 2 schematically shows that a series of physicians can input the symptom list S in the framework of a treatment of a patient A. A set of diagnostic questions F (diagnostic question 1, diagnostic question 2, . . . , diagnostic question k) is generated based on the acquired symptom list S. An association between the respective symptom S or the symptom list S and the respective set of diagnostic questions F is stored in a rule base. This association can be modified by an authorized person.

A particular advantage of the inventive procedure is to be seen in that the set of diagnostic questions F is standardized and thus is accessible to a persistent quality control. Individual failures by physicians that are based on the fact that, for example, the response to a diagnostic question F is forgotten given a manual input can be safely avoided. Moreover, a quality standard at the highest and up-to-date level can be ensured.

For example, in the rule database a specific MR workflow is stored as a function of the symptom list S and the diagnostic questions F or, respectively, their answers A. Moreover, corresponding further workflows can be stored, for example a CT workflow or a US workflow, etc. The lists of the stored workflows can be arbitrarily expanded.

It is typically provided that at least one initial symptom list S has been manually input by an authorized physician. A set of diagnostic questions F that is associated with the list of input symptoms S is thereupon generated automatically or semi-automatically by the system. If the symptom list S includes multiple symptoms, it may be possible that a number of different sets of diagnostic questions F must also be answered. The response to the diagnostic questions F ensues via a corresponding user interface. This can ensue either through a manual input or can be standardized by a selection (menu) of responses A offered to-the user. So that a complete response can be ensured, it is advantageously provided that all diagnostic questions F must first be answered before the system proceeds to the next step. Missing inputs can thus be safely precluded. This increases the safety of the system.

In a preferred embodiment of the invention, it is sets that concern the cases are stored in the databank DB in the event that it is not possible to acquire or to input an initial symptom list. S. This is the case, for example, when the patient is unconscious. In this case, for example, the following association can be stored in the databank DB: “Symptom: ‘Patient is unconscious’>Implementation of an examination with an arbitrary imaging modality”. This ensures that a workflow can be generated even given a missing initial symptom list S. This workflow is unavoidably relatively nonspecific and requires the submission of further examination results for further specification or, respectively, delimitation.

One advantage of the inventive solution described herein is that the relevant data (in particular the necessary documents) are structured and organized optimized with regard to the treatment of the patient. It is thus automatically provided that data sets that are relevant only in other cases but not in the present treatment case of the patient are also not displayed. This distinctly increases the transparency of the system. For example, specific models or templates are thus only shown or displayed when they are necessary in the case of the patient. For example, a template for input of an ultrasound finding is also only output when a ultrasound examination has also actually been executed in the current case.

All data sets that are relevant with regard to a patient are stored in electronic form. This advantageously ensues in the databank DB. This has the advantage that the previous data sets can be accessed without a new input being necessary at a later point of time given a further treatment of the same patient. Moreover, it is possible that already-acquired data sets are automatically relayed to further instances I (for example to a further hospital for implementation of rehabilitation measures) without the instance I explicitly requesting this.

In principle the system is based on the initial inputs of the emergency physician in the form of the initial symptom list S. With regard to the acquisition of the initial symptom list S, it is to be taken into account that this process normally ensues under an enormous time pressure in an exceptional situation. It is therefore possible that the initial symptom list S is either incomplete or contains errors. In an embodiment of the invention, the initial symptom list S is therefore subjected to a check. The check normally ensues at a later point in time by an authorized physician and can, if applicable, lead to the situation that the initial symptom list S is modified, for example by the modification or addition of further individual symptoms. In the event that the initial symptom list S has been modified, in the following method the modified symptom list S is always accessed. It is in particular provided that the (incorrect) initial symptom list S is overwritten with the (error-free) modified symptom list S and thus is stored in the databank DB.

The inventive central control device has interfaces for data exchange with all instances 1. These can be radio interfaces. The emergency physician instances I are advantageously equipped with mobile electronic dynamic standby capability that involves a corresponding radio interface with the central control instance. These devices can be smart cards, mobile telephones or other mobile data media. Such devices are commercially available, and modified devices that have been expanded with a workflow module can be used that are designed for acquisition of parameters with regard to a treatment of the patient, in particular for acquisition of symptoms, patient-specific information such as the existence of allergies, or other parameters. Moreover, the workflow module includes authentication and/or authorization mechanisms for a secure access of the user to the central control device. These authorization mechanisms ensure that the respective user of the electronic device is also authorized for input of the data.

In a preferred embodiment of the invention the interface between the mobile device of the physician (in particular the emergency physician) and the central control device is fashioned one-sided and permits only the transmission of relevant information (in particular of symptoms of the patient) to the central control instance. A retrieval of data from the databank DB or from the central control device is not possible in this case. This increases the security of the system. Alternatively the interface can be bi-directional so as to also permit a data exchange from the central control device to the mobile device.

The invention also concerns a system for generation of a workflow for a medical treatment of a patient, the system having a number of mobile electronic devices that are fashioned with an interface with the central control device and with a central control device that comprises a user interface and which can access a databank DB. The central control device is equipped with means for execution of the method described in the preceding. The system can be dynamically expanded at any arbitrary point in time in that further instances I can be connected to the central control device.

In advantageous development of the inventive system, this can be equipped with further modules
for the purpose of a cost evaluation and prediction,
for the purpose of a planning,
for the purpose of an error detection,
for the purpose of a quality assurance,
for implementation of clinical studies and
for application of the acquired data for other purposes.

For these purposes the inventive system can be equipped with further monitoring modules and communication devices such as, for example, with RFID components, cameras, LED displays, Bluetooth interfaces, infrared interfaces, interfaces for cards from the health care system (insurance cards) etc. With the inventively acquired data it is also possible to not only control the planning of the treatment workflow of a patient in a simple manner but rather to provide consultation services that are setup differently overall for the respective clinical facility E, which consultation services concern, for example, an optimization of the workflow. Moreover, the data for planning of the computer-aided network are used, in particular for planning of the HIS/RIS network (HIS: hospital information system, RIS: radiology information system).

A series of advantages result with the inventive solution:
a higher throughput can be ensured in the respective clinical facility;
costs can be saved in that the personnel who were previously necessary for planning of a treatment schematic can now be spared due to the automated procedure;
moreover, a cost reduction is possible due to the optimized procedure;
an improved and faster medical offering can be ensured; and
a better utilization can be achieved for the instances of the overall system.

Moreover, the significant advantage standing at the forefront for the patient results that a minimal diagnosis time can be ensured while the danger of errors due to incorrect or wrong inputs is reduced.

In the preferred embodiment, the entire method ensues automatically until a manual or semi-automatic input of an initial symptom list S and a manual or semi-automatic input of a response A to the respective data format F. All further method steps, in particular the generation of the workflow, ensue automatically.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for generating a workflow for a medical treatment, comprising a plurality of different treatment segments, of a patient in a medical system, said workflow comprising a plurality of instances for implementation of the respective treatment segments comprising the steps of:
acquiring at least one initial symptom list of a patient;
in a computerized system, automatically generating at least one diagnostic question based on said at least one initial symptom list;
executing at least one medical examination to answer the generated diagnostic question;
acquiring a response to the diagnostic question as a result of execution of said at least one medical examination; and
using said computerized system, at least semi-automatically generating an optimized workflow based on at least one of said initial symptom list and said response, said workflow comprising a sequence of said treatment segments optimized for said patient and including automatic triggers for said treatment segments and checks for availability of respective instances for implementation of said respective treatment segments.

2. A method as claimed in claim 1 comprising at least semi-automatically controlling execution of said sequence in said workflow.

3. A method as claimed in claim 1 comprising displaying at least a portion of the generated workflow in visible form at a user interface.

4. A method as claimed in claim 1 comprising electronically storing at least a portion of the generated workflow in a databank.

5. A method as claimed in claim 4 comprising additionally storing at least a portion of results of execution of the workflow with respect to said patient in said databank.

6. A method as claimed in claim 1 comprising controlling generation of said workflow and execution thereof in a centralized, hierarchically-subordinate control instance including data exchange with associated instances in said workflow.

7. A method as claimed in claim 1 comprising allowing dynamic expansion of said initial symptom list.

8. A method as claimed in claim 1 comprising generating said workflow using a self-teaching algorithm.

9. A method as claimed in claim 1 comprising, for generating said workflow, accessing a central databank at which said treatment segments, results of said treatment segments and intermediate results of said treatment segments are stored.

10. A method as claimed in claim 9 comprising generating prompts for inclusion in said workflow based on said access to said central databank.

11. A centralized control device for generating a workflow for a medical treatment, comprising a plurality of different treatment segments, of a patient in a medical system, said workflow comprising a plurality of instances for implementation of the respective treatment segments, said centralized control device comprising:
an input unit that acquires at least one initial symptom list of a patient;
a computerized system connected to said input unit that automatically generates at least one diagnostic question based on said at least one initial symptom list, and that causes at least one medical examination to be executed to answer the generated diagnostic question, and that acquires a response to the diagnostic question as a result of execution of said at least one medical examination, and that automatically generates an optimized workflow based on at least one of said initial symptom list and said response, said workflow comprising a sequence of said treatment segments optimized for said patient and including automatic triggers for said treatment segments and checks for availability of respective instances for implementation of said respective treatment segments.

12. A centralized control device as claimed in claim 10 comprising an execution unit that executes said workflow.
A computer-readable medium encoded with a data structure, said medium being loadable into a computerized system for generating a workflow for a medical treatment, comprising a plurality of different treatment segments, of a patient in a medical system, said workflow comprising a plurality of instances for implementation of the respective treatment segments, said computerized system being supplied with at least one initial symptom list of a patient, and said data structure programming said computerized system to:
automatically generate at least one diagnostic question based on said at least one initial symptom list;
cause at least one medical examination to be executed to answer the generated diagnostic question;
acquire a response to the diagnostic question as a result of execution of said at least one medical examination; and
generate an optimized workflow based on at least one of said initial symptom list and said response, said workflow comprising a sequence of said treatment segments optimized for said patient and including automatic triggers for said treatment segments and checks for availability of respective instances for implementation of said respective treatment segments.

13. A medical system comprising:
a plurality of medical instances that interact with a patient to perform a medical function selected from the group consisting of treating the patient and examining the patient; and
a centralized control device connected to said plurality of medical instances, said centralized control device generating a workflow for a medical treatment, comprising a plurality of treatment segments at said plurality of instances, said centralized control device acquiring at least one initial symptom list of a patient, automatically generating at least one diagnostic question based on said at least one initial symptom list, executing at least one medical examination to answer the generated diagnostic question acquiring a response to the diagnostic question as a result of execution of said at least one medical examination, and generating an optimized workflow based on at least one of said initial symptom list and said response, said workflow comprising a sequence of said treatment segments optimized for said patient and including automatic triggers for said treatment segments and checks for availability of respective instances for implementation of said respective treatment segments.
